(19) United States
(12) Patent Application Publication
(10) Pub. No.: US 2023/0041497 A1
(43) Pub. Date: Feb. 9, 2023

(54) MOOD ORIENTED WORKSPACE

(71) Applicant: Sony Interactive Entertainment Inc., Tokyo (JP)

(72) Inventors: Steven Osman, San Mateo, CA (US); Mahdi Azmandian, San Mateo, CA (US); Gary Yuan, Hayward, CA (US); James Talmich, Whittier, CA (US); Olga Rudi, San Francisco, CA (US)

(21) Appl. No.: 17/392,764

(22) Filed: Aug. 3, 2021

(51) Int. Cl. G06F 11/34; A61B 5/16; G06F 3/01

(52) U.S. Cl. CPC G06F 11/3438; A61B 5/165; G06F 11/3466; G06F 3/015

(57) ABSTRACT

A system detects a user's mood and in response establishes computer settings including computer game settings, recommends social network interactions, advises other users, alters task scheduling, and in general enhances collective group mood, collective productivity, social interaction, and engagement.

### FIELD

The present application relates to technically inventive, non-routine solutions that are necessarily rooted in computer technology and that produce concrete technical improvements.

### BACKGROUND

As understood herein, the lack of awareness we have towards other people's emotional state during work hours can pose problems. This is especially relevant with work from home and in an increasingly globalized/mobile workforce or collaboration across locations as we do not have access to any social observations or body language. As a result, we assume that everyone is in their best state and expect a certain type of behavior when that might not be the case. Everyone wants to be connected but a lot of the times it is hard to make the first step.

### SUMMARY

A processor-executed application (“app”) makes it fun and easy for people to reach out to one another, make new connections and express support for others including in a computer gaming setting such as for gamers in a gaming community. The app is tailored to each user individually helping them feel emotionally and physically better on any given day. The app allows a user to change and adjust his or her workspace based on the user's current mood. The user selects a mood from available presets on a device (e.g., phone or a personal computer) and the computer workspace establishes wallpaper, music recommendations, dark/light themes, priority contacts, etc. accordingly. Further, the information of the user's mood is visible to anyone who is using the app, but the user can choose whether he or she is open for someone to reach out and talk or not. As a junior or new member of the team, it might be harder to reach out to people directly. Using this app and choosing that the user feels stuck or frustrated might encourage other members of the team or company to reach out to the user and/or make a connection. Alternatively, the user can choose to reach out to someone if they are marked as available. If the user is unable to reach out but wants to show support and/or encouragement, the user has an option to send a preset message, video, e-card, recommendations to share certain social media content, or animated emoji to another user.

Accordingly, a device includes at least one computer storage that is not a transitory signal and that in turn includes instructions executable by at least one processor to identify a mood of a user and establish a workspace of a computer associated with the user based on the mood.

In some examples, the instructions may be executable to identify the mood of the user at least in part by presenting a query on the computer prompting the user to indicate the mood. In other examples, the instructions can be executable to identify the mood of the user at least in part by analyzing performance of the user in using the computer. In still other implementations, the instructions can be executable to identify the mood of the user at least in part by identifying an activity pattern of the user, identifying the closest activity pattern of at least one other person to the activity pattern of the user, and correlating the closest activity pattern of at least one other person to a mood. In yet other embodiments the instructions are executable to identify the mood of the user at least in part by correlating at least one signal representing a biometric parameter of the user to a mood.

In example embodiments, the workspace includes one or more of at least one friend list sorted according to the mood, at least one computer feed such as a news feed, activity feed, social network data feed, sorted according to the mood, at least one calendar altered according to the mood, at least one task list sorted according to the mood.

In some implementations the instructions may be executable to illuminate at least one light based on the mood to indicate to onlookers the mood of the user. In examples, the instructions are executable to present on the computer an indication of at least one person to contact based on the mood.

In another aspect, a method includes determining a mood of a user, and altering a workspace of a computer associated with the user based on the mood.

In another aspect, an assembly includes at least one computer that includes at least one processor programmed with instructions to establish on the computer a workspace including plural workspace features or characteristics. The instructions are executable to alter at least one of the workspace characteristics based at least in part on a mood of a user of the computer.

The computer may be a video game computer and the workspace may be a video game workspace.

The details of the present application, both as to its structure and operation, can best be understood in reference to the accompanying drawings, in which like reference numerals refer to like parts, and in which:

### BRIEF DESCRIPTION OF THE DRAWINGS

FIG. 1 is a block diagram of an example system including an example in accordance with present principles;

FIG. 2 illustrates example overall logic in example flow chart format;

FIG. 3 illustrates an example workspace desk top screen with example parameters that can be tailored to the mood of the user;

FIG. 4 illustrates example logic according to a first technique in example flow chart format for identifying the user's mood;

FIG. 5 shows an example screen shot of a welcome screen related to the technique of FIG. 4;

FIGS. 6 and 7 show example screens shots of example moods a user can select;

FIGS. 8 and 9 illustrate example response screenshots to selecting a mood in

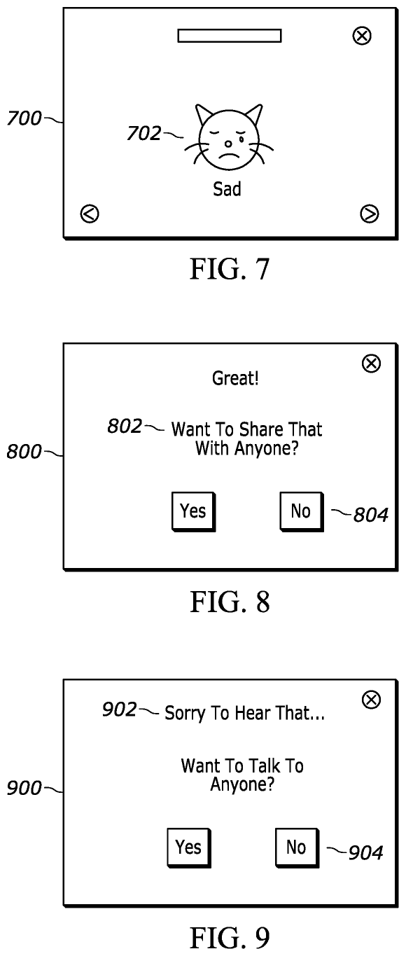

FIGS. 6 and 7, respectively;

FIG. 10 illustrates a screen shot showing an example of list of people and their mood with availability;

FIG. 11 illustrates example logic in example flow chart format of a second mood identification technique;

FIG. 12 illustrates example logic in example flow chart format of a third mood identification technique;

FIG. 13 illustrates example logic in example flow chart format of a fourth mood identification technique;

FIG. 14 illustrates example logic in example flow chart format for enhancing collective mood;

FIG. 15 illustrates example logic in example flow chart format for enhancing collective productivity based on mood; and

FIG. 16 illustrates example logic in example flow chart format for enhancing social interaction.

### DETAILED DESCRIPTION

This disclosure relates generally to computer ecosystems including aspects of consumer electronics (CE) device networks such as but not limited to computer game networks. A system herein may include server and client components which may be connected over a network such that data may be exchanged between the client and server components. The client components may include one or more computing devices including game consoles such as Sony PlayStation® or a game console made by Microsoft or Nintendo or other manufacturer, virtual reality (VR) headsets, augmented reality (AR) headsets, portable televisions (e.g., smart TVs, Internet-enabled TVs), portable computers such as laptops and tablet computers, and other mobile devices including smart phones and additional examples discussed below. These client devices may operate with a variety of operating environments. For example, some of the client computers may employ, as examples, Linux operating systems, operating systems from Microsoft, or a Unix operating system, or operating systems produced by Apple, Inc., or Google. These operating environments may be used to execute one or more browsing programs, such as a browser made by Microsoft or Google or Mozilla or other browser program that can access websites hosted by the Internet servers discussed below. Also, an operating environment according to present principles may be used to execute one or more computer game programs.

Servers and/or gateways may include one or more processors executing instructions that configure the servers to receive and transmit data over a network such as the Internet. Or a client and server can be connected over a local intranet or a virtual private network. A server or controller may be instantiated by a game console such as a Sony PlayStation®, a personal computer, etc.

Information may be exchanged over a network between the clients and servers. To this end and for security, servers and/or clients can include firewalls, load balancers, temporary storages, and proxies, and other network infrastructure for reliability and security. One or more servers may form an apparatus that implement methods of providing a secure community such as an online social website to network members.

A processor may be a single- or multi-chip processor that can execute logic by means of various lines such as address lines, data lines, and control lines and registers and shift registers.

Components included in one embodiment can be used in other embodiments in any appropriate combination. For example, any of the various components described herein and/or depicted in the Figures may be combined, interchanged, or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

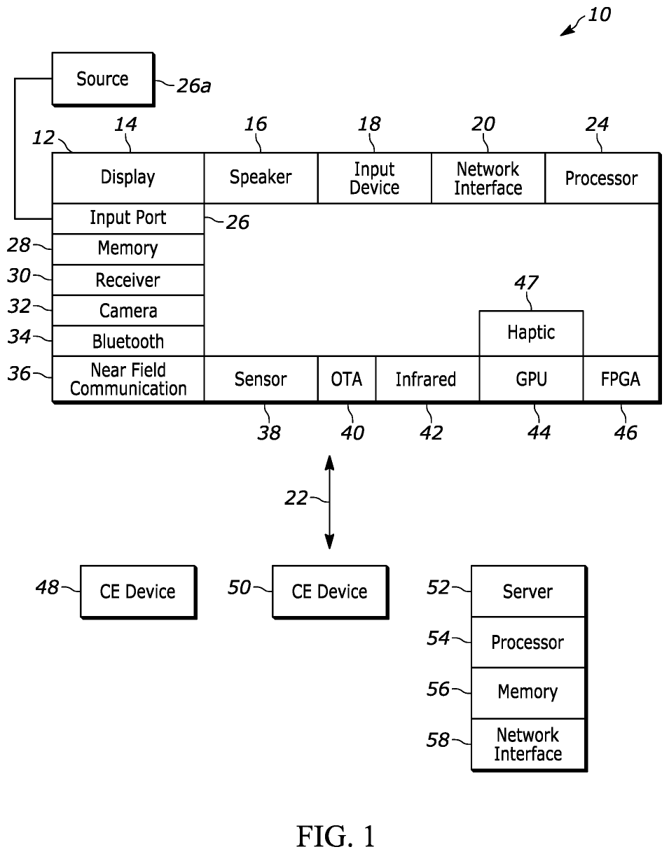

Now specifically referring to FIG. 1, an example system 10 is shown, which may include one or more of the example devices mentioned above and described further below in accordance with present principles. The first of the example devices included in the system 10 is a consumer electronics (CE) device such as an audio video device (AVD) 12 such as but not limited to an Internet-enabled TV with a TV tuner (equivalently, set top box controlling a TV). The AVD 12 alternatively may also be a computerized Internet enabled (“smart”) telephone, a tablet computer, a notebook computer, a HMD, a wearable computerized device, a computerized Internet-enabled music player, computerized Internet-enabled headphones, a computerized Internet-enabled implantable device such as an implantable skin device, etc. Regardless, it is to be understood that the AVD 12 is configured to undertake present principles (e.g., communicate with other CE devices to undertake present principles, execute the logic described herein, and perform any other functions and/or operations described herein).

Accordingly, to undertake such principles the AVD 12 can be established by some, or all of the components shown in FIG. 1. For example, the AVD 12 can include one or more displays 14 that may be implemented by a high definition or ultra-high definition “4K” or higher flat screen and that may be touch-enabled for receiving user input signals via touches on the display. The AVD 12 may include one or more speakers 16 for outputting audio in accordance with present principles, and at least one additional input device 18 such as an audio receiver/microphone for entering audible commands to the AVD 12 to control the AVD 12. The example AVD 12 may also include one or more network interfaces 20 for communication over at least one network 22 such as the Internet, an WAN, an LAN, etc. under control of one or more processors 24. Thus, the interface 20 may be, without limitation, a Wi-Fi transceiver, which is an example of a wireless computer network interface, such as but not limited to a mesh network transceiver. It is to be understood that the processor 24 controls the AVD 12 to undertake present principles, including the other elements of the AVD 12 described herein such as controlling the display 14 to present images thereon and receiving input therefrom. Furthermore, note the network interface 20 may be a wired or wireless modem or router, or other appropriate interface such as a wireless telephony transceiver, or Wi-Fi transceiver as mentioned above, etc.

In addition to the foregoing, the AVD 12 may also include one or more input and/or output ports 26 such as a high-definition multimedia interface (HDMI) port or a USB port to physically connect to another CE device and/or a headphone port to connect headphones to the AVD 12 for presentation of audio from the AVD 12 to a user through the headphones. For example, the input port 26 may be connected via wire or wirelessly to a cable or satellite source 26a of audio video content. Thus, the source 26a may be a separate or integrated set top box, or a satellite receiver. Or the source 26a may be a game console or disk player containing content. The source 26a when implemented as a game console may include some or all of the components described below in relation to the CE device 48.

The AVD 12 may further include one or more computer memories 28 such as disk-based or solid-state storage that are not transitory signals, in some cases embodied in the chassis of the AVD as standalone devices or as a personal video recording device (PVR) or video disk player either internal or external to the chassis of the AVD for playing back AV programs or as removable memory media or implemented by the below-described server. Also, in some embodiments, the AVD 12 can include a position or location receiver such as but not limited to a cellphone receiver, GPS receiver and/or altimeter 30 that is configured to receive geographic position information from a satellite or cellphone base station and provide the information to the processor 24 and/or determine an altitude at which the AVD 12 is disposed in conjunction with the processor 24. The component 30 may also be implemented by an inertial measurement unit (IMU) that typically includes a combination of accelerometers, gyroscopes, and magnetometers to determine the location and orientation of the AVD 12 in three dimensions. The component 30 include or be instantiated by a camera of event-based sensor.

Continuing the description of the AVD 12, in some embodiments the AVD 12 may include one or more cameras 32 that may be a thermal imaging camera, a digital camera such as a webcam, and/or a camera integrated into the AVD 12 and controllable by the processor 24 to gather pictures/images and/or video in accordance with present principles, and/or an event-based sensor. Also included on the AVD 12 may be a Bluetooth transceiver 34 and other Near Field Communication (NFC) element 36 for communication with other devices using Bluetooth and/or NFC technology, respectively. An example NFC element can be a radio frequency identification (RFID) element.

Further still, the AVD 12 may include one or more auxiliary sensors 38 (e.g., a motion sensor such as an accelerometer, gyroscope, cyclometer, or a magnetic sensor, an infrared (IR) sensor, an event-based sensor, an optical sensor, a speed and/or cadence sensor, a gesture sensor (e.g., for sensing gesture command), providing input to the processor 24. The AVD 12 may include an over-the-air TV broadcast port 40 for receiving OTA TV broadcasts providing input to the processor 24. In addition to the foregoing, it is noted that the AVD 12 may also include an infrared (IR) transmitter and/or IR receiver and/or IR transceiver 42 such as an IR data association (IRDA) device. A battery (not shown) may be provided for powering the AVD 12, as may be a kinetic energy harvester that may turn kinetic energy into power to charge the battery and/or power the AVD 12. A graphics processing unit (GPU) 44 and field programmable gated array 46 also may be included. One or more haptics generators 47 may be provided for generating tactile signals that can be sensed by a person holding or in contact with the device.

Still referring to FIG. 1, in addition to the AVD 12, the system 10 may include one or more other CE device types. In one example, a first CE device 48 may be a computer game console that can be used to send computer game audio and video to the AVD 12 via commands sent directly to the AVD 12 and/or through the below-described server while a second CE device 50 may include similar components as the first CE device 48. In the example shown, the second CE device 50 may be configured as a computer game controller manipulated by a player or a head-mounted display (HMD) worn by a player. In the example shown, only two CE devices are shown, it being understood that fewer or greater devices may be used. A device herein may implement some or all of the components shown for the AVD 12. Any of the components shown in the following figures may incorporate some or all of the components shown in the case of the AVD 12.

Now in reference to the afore-mentioned at least one server 52, it includes at least one server processor 54, at least one tangible computer readable storage medium 56 such as disk-based or solid-state storage, and at least one network interface 58 that, under control of the server processor 54, allows for communication with the other devices of FIG. 1 over the network 22, and indeed may facilitate communication between servers and client devices in accordance with present principles. Note that the network interface 58 may be, e.g., a wired or wireless modem or router, Wi-Fi transceiver, or other appropriate interface such as, e.g., a wireless telephony transceiver.

Accordingly, in some embodiments the server 52 may be an Internet server or an entire server “farm” and may include and perform “cloud” functions such that the devices of the system 10 may access a “cloud” environment via the server 52 in example embodiments for, e.g., network gaming applications. Or the server 52 may be implemented by one or more game consoles or other computers in the same room as the other devices shown in FIG. 1 or nearby.

The components shown in the following figures may include some or all components shown in FIG. 1. The techniques described herein may be embodied in a computer application (“app”) downloaded from server storages on the Internet and stored on the implementing computer on any of the storages described herein.

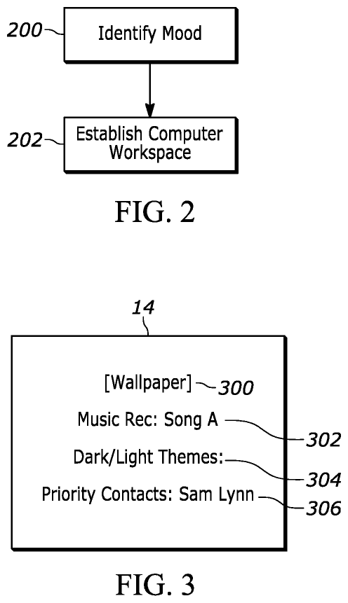

FIG. 2 illustrates overall logic. The mood of a user is identified at block 200, and at block 202 the computer workspace of a computer and/or video game component such as a game console associated with the user is established based on the mood identified at block 200. The workspace may include a computer game space.

An example workspace, the features of which may be established according to the mood of the user, and which may change as the user's mood changes, is shown in FIG. 3. As shown, the example workspace may be an audio-video computer presentation presented on any display herein, such as the display 14 shown in FIG. 1. Any or all of the example components of the workspace may vary according to user mood.

The workspace may include, e.g., background wallpaper 300 such as a colored pattern, photo, or other image, etc. The workspace may include and/or play a song recommendation 302. The workspace may further include dark/light themes 304 and a list 306 of contacts, the priority among which may be established by the mood such that, for example, “Sam” may be a higher priority contact than “Lynn” when the mood is upbeat, and “Lynn” may be a higher priority contact than “Sam” when the mood is otherwise than upbeat.

Other workspace features that may be changed based on mood can include audio volume. A louder volume may be more appropriate for a person who is in a good mood and a lower volume established for the same person in a bad mood, or vice-versa. Font size may change so that a person in a good mood may be presented smaller font and that same person in a bad mood may be presented larger font, or vice-versa.

Yet again, when a person is in a good mood, thumbnail images of certain friends may be displayed larger than they would be if the person is in a bad mood, and vice-versa.

These workspace or game space features may be established automatically based on mood, or suggestions may simply appear on screen for the person to make the noted changes.

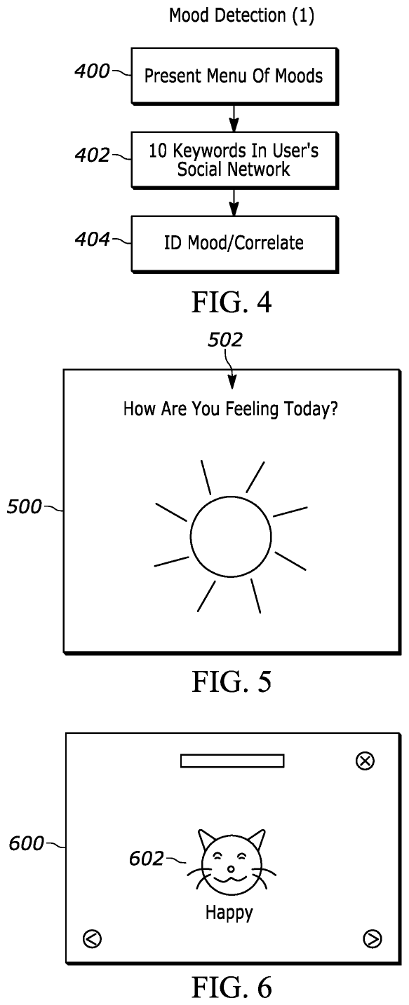

Mood can be identified using various techniques. FIG. 4-1 illustrates a first technique in which the user is asked his or her mood. Commencing at block 400 in FIG. 4, a menu or other selection structure may be presented on the user's display listing moods from whence the user may elect an appropriate input. Furthermore, at block 402 keywords may be identified from, e.g., the user's social network entries that indicate mood, e.g., happiness, sadness, anxious, etc. Block 404 indicates that the user's mood is identified by one or more of the user's selection at block 400 and social network entries at block 402 and correlated to workspace features for implementation of the mood-appropriate workspace on the user's computer.

The logic of FIG. 4 can be implemented at the start of the workday, followed by another mid-day check-in.

The principles of FIG. 4 are illustrated in FIGS. 5 et seq. (which may also illustrate correlating mood to workspace environment when other mood identification techniques described herein are used). FIGS. 5-7 illustrate that instead of a drop-down menu listing moods, a user interface (UI) 500 may be presented asking at 502 what the user's mood is. A UI 600 may be presented in FIG. 6 indicating at 602 the mood “happy” while a UI 700 may be presented as shown in FIG. 7 indicating at 702 the mood “sad”. The UIs 500, 600, 700 shown in FIGS. 5-7 may be presented as a single UI.

FIG. 8 illustrates a UI 800 that may be presented responsive to the user selecting “happy” from the UI 600 in FIG. 6. The UI 800 may include a prompt 802 asking the user if the user would like to share the mood indication with others, and the user can select a yes or no selector 804 accordingly. Selecting “yes” means that the user's computers will send an indication of the user's mood to other users such as friends of the user, co-workers, family members, etc. as may be chosen by the user.

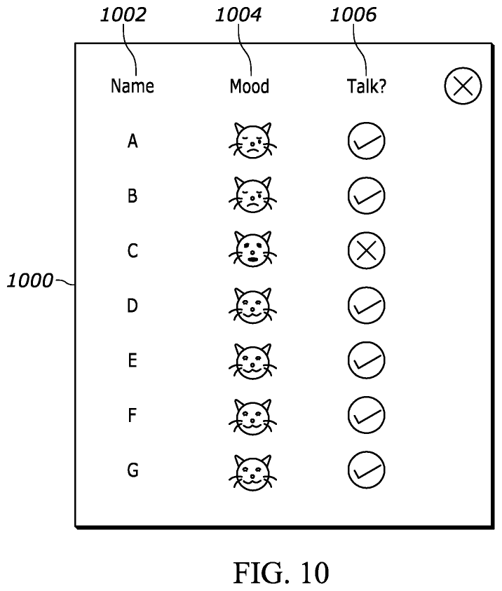

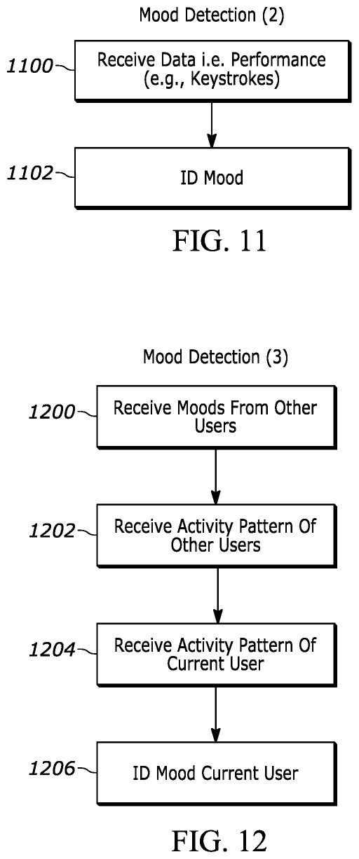

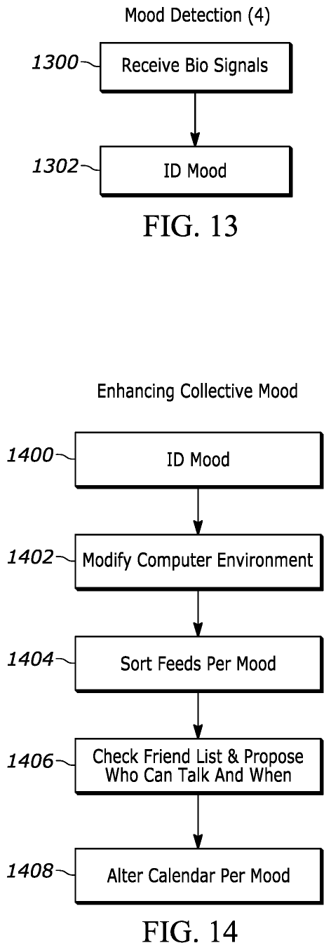

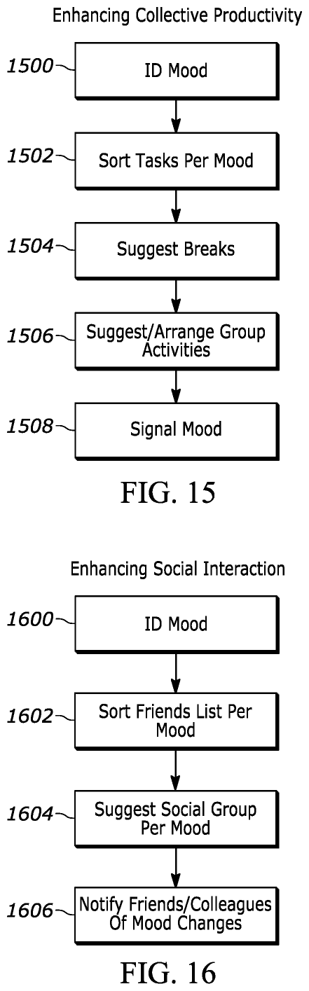

FIG. 9 illustrates a UI 900 that may be presented responsive to the user selecting “sad” from FIG. 7. A prompt 902 may be presented for the user to select, using a selector 904, whether the user would like to talk to anyone. Selecting “no” ca result in the user's computer passing phone calls through to voice mail, for example, and/or delaying presenting text or email messages or other attempts to contact the user. Selecting “yes” may result in the computer passing communications through to the user and may also result in the computer automatically calling a contact or otherwise communicating the user's mood to a contact designated by the user for situations in which the user is “sad”, so that the user may be given immediate support.

FIG. 10 illustrates further. A UI 1000 may present a list 1002 of persons registered with a list 1004 of moods correlating each person in the person list 1002 as a contact for a particular user mood in the mood list 1004. The user may select in a column 1006 whether each person in the person list 1002 is to be contacted or not (and/or whether calls from the respective person are to be passed through the user's computer to the user) depending on the respective mood of the selecting user in the mood list 1004.

FIGS. 11-13 illustrate alternative techniques for identifying user mood at block 200 in FIG. 2. These techniques may be rule-based and/or may employ machine learning (ML) models that essentially train on training data sets that correlate the parameters discussed in each technique with ground truth mood.

Accordingly, present principles may employ machine learning models, including deep learning models. Machine learning models use various algorithms trained in ways that include supervised learning, unsupervised learning, semi-supervised learning, reinforcement learning, feature learning, self-learning, and other forms of learning. Examples of such algorithms, which can be implemented by computer circuitry, include one or more neural networks, such as a convolutional neural network (CNN), recurrent neural network (RNN) which may be appropriate to learn information from a series of images, and a type of RNN known as a long short-term memory (LSTM) network. Support vector machines (SVM) and Bayesian networks also may be considered to be examples of machine learning models.

As understood herein, performing machine learning involves accessing and then training a model on training data to enable the model to process further data to make predictions. A neural network may include an input layer, an output layer, and multiple hidden layers in between that that are configured and weighted to make inferences about an appropriate output.

FIG. 11 illustrates that mood can be identified by analyzing performance of the user. Commencing at block 1100, data is received of the user's performance. This data may include, e.g., rate of keystrokes (higher indicating more alert), rate of web surfing (higher indicating distracted), eye movement (steady gaze as imaged by a camera indicating focused), etc. The data received at block 1100 may be compared to historical performance data for that user to determine mood at block 1102, e.g., whether the user is behaving more sluggish than usual or is being particularly efficient. Performance of the user can also include accuracy, e.g., how often they press undo or backspace, how often they fail/retry something.

FIG. 12 illustrates a technique for identifying user mood based on usage patterns of other users. Commencing at block 1200, indications of ground truth moods of other users are received along with their contemporaneous activity patterns at block 1202. The activity pattern of the current user is received at block 1204 and the mood of the current user identified at block 1206 by comparing the current user's usage pattern from block 1204 to the activity patterns of other users from block 1202 and correlating the nearest matching pattern at block 1202 to the corresponding mood from block 1200.

Thus, the technique of FIG. 12 creates a classification and clustering algorithm based on activity patterns and data from other users. As an example, in the workplace, by analyzing others it may be noted that they classify themselves as ‘anxious’ they are more likely to conduct reading/research activities, and when they are ‘happy’ they are more likely to tackle challenging bugs. Similar examples can be drawn from gaming patterns, for instance, the likelihood that a player will take on a long mission, simple challenges, puzzle versus action, and so on.

FIG. 13 illustrates that user mood may be identified at block 200 in FIG. 2 using biometric and other physical attributes, for instance, their heart rate, galvanic skin response, blood pressure, pitch of voice, word selections and speed of speaking, signals indicating such biometric attributes being received at block 1300. This can also include blink rate, eye motion, eye openness, pupil dilation and redness in eye blood vessels. Based on the biometric signals, mood of the user is identified at block 1302. The technique of FIG. 13 may be especially suited for a ML model trained on a training set that correlates ground truth mood to one or more biometric signal attributes. Once biometric signals have been correlated to mood, in some embodiments future mood-based changes to the workspace may be made automatically based on the future biometric signals so the user does not have to be asked his or her mood.

A wearable device such as a wristwatch can provide information to correlate its output to prior mood. For example, a wearable device that includes biometric sensors may be used in connection with FIG. 13.

FIG. 14 illustrates a technique for enhancing the collective mood of multiple users. Commencing at block 1400, the mood(s) of one or more users are identified using any of the techniques for example described herein. FIG. 14 may be used, e.g., for a large network of computer game players and thus can apply to a workspace or game space as well.

Moving to block 1402, each user's environment can be modified and tailored to enhance their mood. For instance, if a person is feeling depressed, they can be presented themes on their respective computing devices (images, backgrounds, color schemes, music, notification sounds, IOT devices color) to lift their spirits. Similarly, if a person is feeling happy, that person's good mood can be boosted by providing highly energetic themes to capitalize on this.

Further, proceeding to block 1404, based on the person's mood and likelihood to be receptive of certain news, tasks, challenges, etc., their feeds (news feeds, gaming feeds, even email sorting) can be sorted in such a way as to present the data most likely to work well given their current mood. This sorting may be amenable to ML modeling trained on ground truth data correlating mood to feed sort.

Similarly, proceeding to block 1406 when a user's mood is down, the user's computer or other computer can automatically check their friend's list to find who is available to talk, then propose a time to meet based on their availabilities as indicated by, e.g., electronic schedules. At block 1408 the user's calendar may be altered based on the user's mood. If a user's mood is down for example, the user's calendar can be altered to blocking out the user's time which would otherwise indicate “available” by, for instance, entering a tentative meeting to ensure coworkers will consult the user before scheduling time in their calendar.

FIG. 15 illustrates techniques for enhancing collective productivity based on the mood(s) of one or more users, which are identified at block 1500. Moving to block 1502, tasks can be sorted given ones that are most appropriate for a person's mood. Tasks can include, work related tasks, personal TODO lists, game recommendations, and even specific activities within a game.

Proceeding to block 1504, appropriate breaks can be suggested, for instance, work-out breaks, lunch breaks, social time (or personal time) to improve productivity and gaming efficiency. Similarly, at block 1506 certain group activities can be arranged when they are most likely to succeed. For instance, a user's computer may present a proposal to postpone a critical idea pitch if several the stakeholders are not in a receptive mode. Conversely, an introverted junior player/engineer may be nudged (e.g., by an audio and/or video prompt) towards seeking advice from a more senior player/engineer if the more senior person is in a good mood.

Understanding that mood can change, a person may be monitored as the day proceeds. For example, in the case of a computer game player, the player may be asked for mood updates and/or the player's activities can be monitored and correlated to mood shifts. For example, a longer length of play or work may indicate a change to a better mood, and in the case of a computer game an initially “sad” player might play but then play for a length of time exceeding a threshold, the system can suggest that game to the player in the future when the player is feeling “sad”. Similarly, if a player is aggressive a “good” mood might be inferred while if a player takes numerous or long breaks a distracted mood may be inferred. Productivity and game score may be matched to a first mood so that when the same player in the future exhibits similar productivity or game score, the first mood on the part of the player may be inferred.

An imager on a game console or workstation computer may image a person and computer vision may be employed on the images to ascertain a change of mood. Likewise, if computer vision shows the person constantly looking at a phone, a “distracted” mood may be inferred.

Furthermore, at block 1508 a physical device with a small light can also be placed on the user's desk to signal their mood and availability. A green light, for example, may indicate that the user is in a mood considered to be approachable whereas a red light may signify the opposite.

FIG. 16 illustrates techniques for enhancing social interaction based on the mood(s) of one or more users identified at block 1600. Techniques discussed above may be used to raise the collective mood of a group of users through analysis of the collective state of minds.

Additionally, at block 1602 the friends list of the users in a group may be sorted such that the friends or co-workers a particular user is most likely to get along with appear at the top of the friends list of that particular user. As but one example, friends whose moods are “calm” may be placed at the top of a friend list of a user whose mood is “anxious”. As another example, friends whose moods are “sensitive” may be placed at the top of a friend list of a user whose mood is “sad”. Likewise, when a person is in a bad mood, friends on the list who may be, e.g., loud, or obnoxious can be placed at the bottom of the list. Various heuristics matching moods of friends to the mood of a particular user may be established by experts or by ML models. Social bonds can be further enhanced by suggesting at block 1604 that, for example, a good-mood group try to cheer up particular individuals using audio and/or video prompts on the computers of the people in the group. As indicated at block 1606, when a user's mood improves, their friends who helped them can be notified for positive reinforcement. Colleagues waiting on the user for a task will also be notified to signal it is an appropriate time to be contacted again. Failed/successful interactions can be analyzed to increase the overall effectiveness of the system, driving the collective community to an overall better mood through adjustments to future pairings and suggestions.

The communications of mood information and other information herein may be made via a wide area computer network.

Present techniques may also be used for enhancing engagement. Present principles can lead to improved engagement, whether at work or at play. Because tasks are selected appropriate to people's moods, they are more likely to stick with them and to complete more tasks. They will spend more time working/playing than if they were doing incorrect tasks.

The computer workspace being modified per mood may be a computer game development workspace.

Because environments are appropriate to people's moods, they will feel comfortable/comforted even further boosted by their environment.

Because social recommendations are mood aware, not only for the individual, but for the collective, people are more likely to have successful interactions—or avoid them if they are likely not to succeed. This means the time spent with others (or not) will be more enjoyable and/or less annoying.

It will be appreciated that whilst present principals have been described with reference to some example embodiments, these are not intended to be limiting, and that various alternative arrangements may be used to implement the subject matter claimed herein.

What is claimed is:

1. A device comprising:
at least one computer storage that is not a transitory signal and that comprises instructions executable by at least one processor to:
identify a mood of a user; and
establish a workspace of a computer associated with the user based on the mood.

2. The device of claim 1, comprising the at least one processor.

3. The device of claim 1, wherein the instructions are executable to:
identify the mood of the user at least in part by presenting a query on the computer prompting the user to indicate the mood.

4. The device of claim 1, wherein the instructions are executable to:
identify the mood of the user at least in part by analyzing performance of the user in using the computer.

5. The device of claim 1, wherein the instructions are executable to:
identify the mood of the user at least in part by:
identifying an activity pattern of the user;
identifying the closest activity pattern of at least one other person to the activity pattern of the user; and
correlating the closest activity pattern of at least one other person to a mood attributed to the user.

6. The device of claim 1, wherein the instructions are executable to:
identify the mood of the user at least in part by correlating at least one signal representing a biometric parameter of the user to a mood.

7. The device of claim 1, wherein the workspace comprises:
at least one friend list or associate list sorted according to the mood.

8. The device of claim 1, wherein the workspace comprises:
at least one computer feed sorted according to the mood.

9. The device of claim 1, wherein the workspace comprises:
at least one calendar altered according to the mood.

10. The device of claim 1, wherein the workspace comprises:
at least one task list sorted according to the mood.

11. The device of claim 1, wherein the instructions are executable to:
illuminate at least one light based on the mood.

12. The device of claim 1, wherein the instructions are executable to:
present on the computer an indication of at least one person to contact based on the mood.

13. A method comprising:
determining a mood of a user; and
altering a workspace of a computer associated with the user based on the mood.

14. The method of claim 13, comprising determining the mood of the user at least in part by:
presenting a query on the computer prompting the user to indicate the mood.

15. The method of claim 13, comprising determining the mood of the user at least in part by:
analyzing performance of the user in using the computer.

16. The method of claim 13, comprising determining the mood of the user at least in part by:
identifying an activity pattern of the user;
identifying the closest activity pattern of at least one other person to the activity pattern of the user; and
correlating the closest activity pattern of at least one other person to a mood attributed to the user.

17. The method of claim 13, comprising determining the mood of the user at least in part by:
correlating at least one signal representing a biometric parameter of the user to a mood.

18. An assembly comprising:
at least one computer comprising at least one processor programmed with instructions to:
establish on the computer a workspace comprising plural workspace characteristics; and
alter at least one of the workspace characteristics based at least in part on a mood of a user of the computer.

19. The assembly of claim 18, wherein the processor is programmed to:
alter one or more of a video background presented on the computer, room lighting, room temperature, curtain position, background music, activity recommendation based on the mood.

20. The assembly of claim 18, wherein the processor is programmed to:
alter a list of contacts presented on the computer based on the mood.
